(19) United States
(12) Patent Application Publication
(10) Pub. No.: US 2018/0333433 A1
(43) Pub. Date: Nov. 22, 2018

(54) SYNTHESIS OF SILVER NANOPARTICLES USING SESAME OIL CAKE

(71) Applicant: KING SAUD UNIVERSITY, RIYADH (SA)

(72) Inventors: MOHAMAD SALEH ALSALHI, RIYADH (SA); SANDHANASAMY DEVANESAN, RIYADH (SA); AKRAM AHMED ALFURAYDI, RIYADH (SA); MYSOON M.F. AL-ANSARI, RIYADH (SA)

(21) Appl. No.: 15/599,429

(22) Filed: May 18, 2017

(51) Int. Cl. A61K 33/38; A61K 36/185; C11B 3/00; B22F 9/24; B22F 1/00

(52) U.S. Cl. CPC A61K 33/38; A61K 36/185; A61K 47/48884; B82Y 40/00; C11B 3/008; B22F 9/24; B22F 1/0018; A61K 47/481

(57) ABSTRACT

A method of synthesizing silver nanoparticles includes using sesame (Sesamum indicum) oil cake extract as a reducing agent. The silver nanoparticles can range in size from about 6 nm to about 15 nm. The silver nanoparticles can have an average particle size of about 10 nm.

### BACKGROUND OF THE INVENTION

### 1. Field of the Invention

The present subject matter relates to silver nanoparticles and, more particularly, to a method for synthesizing silver nanoparticles (AgNPs) using sesame oil til cake (Sesamum indicum).

### 2. Description of the Related Art

In recent years, the synthesis of nanoparticles using plants, and development of their biological activities, has become increasingly significant in the fields of nano-medicine, pharmaceutical industries, and other related technologies. Silver nanoparticles are increasingly used in the medicinal, pharmaceutical, agricultural, and water purification industries. In particular, silver nanoparticles are increasingly considered for anticancer and antimicrobial activities.

Sesame (Sesamum indicum, L.), is one of the most important crops throughout the world and is mainly cultivated in India, China, Japan, and other East Asian countries. In Saudi Arabia, sesame is cultivated mainly in Makkah and Gizan regions. Sesame seeds have been used as food and to produce an edible oil since ancient times. Sesame seeds provide numerous beneficial effects for human health. The beneficial biological ingredients present in the seed include vitamins, phytosterols, polyunsaturated fatty acids, tocopherols, sesamin and sesamolin.

Recently, many studies have been reported that sesame seeds can reduce hypertension and help prevent various cardiac conditions. Moreover, sesame seeds contain magnesium and other nutrients that have been used to help lower blood pressure and plasma glucose in hypersensitive diabetic patients, and also have been shown to significantly reduce the risk of malignant tumors including colorectal cancer, breast cancer, prostate cancer, and liver cancer.

Sesame oil cake is the protein-rich by-product obtained after sesame seed oil extraction. Sesame oil cake is rich in amino acids and crude proteins. The sesame oil cake contains less aflatoxin than certain alternatives, and provides certain medicinal properties for treating cancer, as well as antioxidant and antifungal effects.

Thus, an environmentally friendly synthesis of silver nanoparticles using sesame (Sesamum indicum) oil cake provides a new, improved solution to certain health issues.

### SUMMARY OF THE INVENTION

The present subject matter relates to the synthesis of silver nanoparticles from sesame (Sesamum indicum) oil til cake, also known as oil cake. The synthesized silver nanoparticles achieve a suitable particle size for biological activity. For example, silver nanoparticles were synthesized having a particle size ranging from about 6.63 nm to about 14.50 nm. The silver nanoparticles synthesized according the present teachings demonstrate cytotoxicity against a human breast cancer cell line (MCF-7 cells).

According to one embodiment, a green method of producing silver nanoparticles using Sesame indicum oil til cake is provided. The method may include: obtaining or producing Sesamum indicum oil til cake that results from an oil-extracting process performed on Sesamum indicum; washing the oil til cake to remove additives from the oil-extracting process; drying the oil til cake that was washed; grinding the dried oil til cake to a fine powder; soaking the oil til cake powder in water; filtering the oil til cake powder to produce sesame oil cake extract; adding water to silver nitrate to obtain a silver nitrate solution; adding the silver nitrate solution to the sesame oil cake extract; and allowing sufficient time for development of the silver nanoparticles.

In the present method, the sesame oil til cake extract reduces silver ions to AgNPs in a rapid and ecofriendly manner. The rapid reduction of silver (Ag+) ions was monitored using UV-vis spectroscopy with different time intervals [48 h to 168 h] which exhibited an absorbance peak at 420 nm. The TEM results exhibited silver nanoparticles having a spherical shape with a diameter ranging from about 6.6 nm to about 14.8 nm.

This is the first known study of synthesizing silver nanoparticles from sesame oil cake, and the cytotoxicity of the resulting silver nanoparticles as applied to human breast cancer cell line (MCF-7). The synthesis method provided herein achieves silver nanoparticles having a particle size diameter that is ideal for biological activity. The cytotoxic effects of the synthesized silver nanoparticles were evaluated with different concentrations, i.e., 2.5 μg/mL, 7.5 μg/mL, and 10 μg/mL, of synthesized AgNPs applied to a human breast cancer cell line (MCF-7 cells). The results of these experiments showed that treatment with AgNPs using sesame oil cake acted to regulate apoptotic induction against MCF-7 cell lines.

These and other features of the present subject matter will become readily apparent upon further review of the following specification and drawings.

### BRIEF DESCRIPTION OF THE DRAWINGS

FIG. 1 shows the UV-visible spectra of 0.5 mM AgNO₃ interacted with sesame oil cake at different time intervals.

FIG. 2 shows a plot of the UV absorbance, at a wavelength of 420 nm, of 0.5 mM AgNO₃ interacted with sesame oil cake at time intervals from 48 h to 168 h.

FIG. 3 shows FTIR spectrum of the AgNPs synthesized using sesame oil cake.

FIG. 4 shows X-Ray Diffraction Analysis (XRD) spectrum of crystalline AgNPs synthesized using sesame oil cake.

FIG. 5 shows a transmission electron microscopy (TEM) image of AgNPs synthesized using sesame oil cake.

FIG. 6 shows Energy Dispersive X-ray diffraction (EDX) images of the synthesized AgNPs.

FIG. 7A shows data for human breast cancer cell line (MCF-7) with no treatment.

FIG. 7B shows data for human breast cancer cell line (MCF-7) treated with the synthesized AgNPs at concentrations of 2.5 μg/mL.

FIG. 7C shows data for human breast cancer cell line (MCF-7) treated with the synthesized AgNPs at concentrations of 7.5 μg/mL.

FIG. 7D shows data for human breast cancer cell line (MCF-7) treated with the synthesized AgNPs at concentrations of 10 μg/mL.

### DETAILED DESCRIPTION OF THE PREFERRED EMBODIMENTS

Reference will now be made in detail to embodiments of the present disclosure, examples of which are illustrated in the accompanying drawings. For purposes of this disclosure, “Sesame indicum oil til cake”, “sesame oil til cake”, and “sesame oil cake” are used interchangeably throughout the disclosure, except as otherwise provided.

Sesame is widely used as an early crop because it has the ability to grow widely in cultivating areas where other crops fail to survive. Accordingly, sesame is known as a “survivor crop”.

Sesame seeds have abundant dietary ingredients, including calcium, magnesium, selenium, zinc, vitamins, etc. Sesame seeds also present several beneficial biological activities, including antihypertensive, anticancer and antimicrobial properties. Sesame oil cake is the protein-rich by-product obtained after sesame seed oil extraction. Sesame oil cake can include about 32% crude protein and 40% amino acids.

According to one embodiment, a green or eco-friendly method of producing silver nanoparticles (AgNPs) using Sesame indicum oil til cake is provided. The method can include obtaining or producing Sesamum indicum oil til cake that results from an oil-extracting process performed on Sesamum indicum. The oil til cake can then be washed to remove additives from the oil-extracting process. After the oil til cake is washed, the oil til cake can be dried. Subsequently, the dried oil til cake can be ground to a fine powder. The finely ground oil til cake powder can be soaked in water and then filtered to produce sesame oil cake extract. In some embodiments, the oil til cake powder is soaked in DH₂O (distilled water).

After the sesame oil cake extract is obtained, a solution of silver nitrate in water can be added to the sesame oil cake extract. In some embodiments, the water used to dissolve the silver nitrate is distilled water. The solution of silver nitrate in water and the sesame oil cake extract can be reacted for a sufficient amount of time to form silver nanoparticles. For example, the silver nanoparticles can develop over a period of time ranging from about 48 hours to 168 hours.

The silver nanoparticles obtained from the sesame oil cake extract can be used to effectively treat cancer. An effective amount of the silver nanoparticles obtained from the above method can be administered to a patient with cancer. In particular, the silver nanoparticles may be administered to a patient being treated for breast cancer. The silver nanoparticles can be used in combination with one or more additional treating agents for cancer.

The following examples illustrate the present teachings.

### Example 1

### Preparation of Sesame Oil Cake Extract

Sesamum indicum oil cake was purchased from Makah region, Kingdom of Saudi Arabia. 100 g of sesame oil cake was washed to manually remove added molasses during the oil-extracting process. The washed cake was completely dried at room temperature, and ground to a fine power. 10 g of sesame oil cake powder was soaked in 200 ml of double distilled water [DH₂O] for 24 h. The next day, the sesame oil cake extract was filtered using Whatman No. 1 filter paper. The sesame oil cake extract had a pale reddish color.

### Example 2

### Nanoparticle Synthesis

The sesame oil cake extract was collected in a conical flask. Separately, 0.5 mM of silver nitrate was dissolved in 250 ml of distilled water, and added to 10 ml of the obtained sesame cake extract. The mixed colorless solution developed a dark brown color over time. The color change was observed from 48 h onwards, with the dark brown color being completely achieved by about 168 h. The color change indicates formation of AgNPs due to reduction of silver ions interacting with active molecules present in the sesame cake extract.

The dark brown color is attributed to surface Plasmon resonance, which is a size-dependent property of nanoparticles. The synthesis process was conducted at room temperature. The remaining amount of sesame oil cake aqueous extract was stored at 4° C. for further analysis.

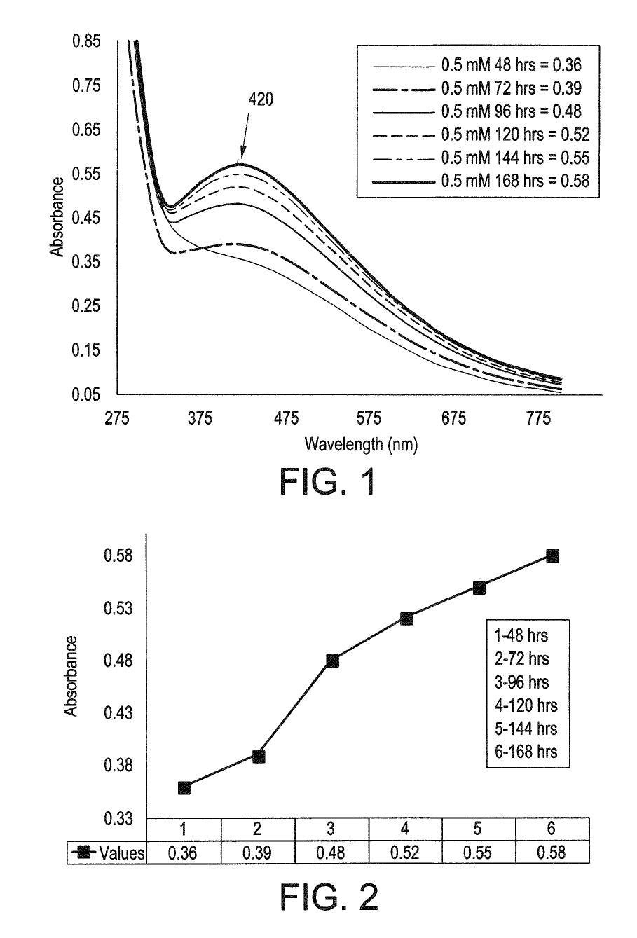

FIG. 1 shows the UV-visible spectra of 0.5 mM AgNO₃ interacted with sesame oil cake at different time intervals. In particular, UV-visible spectroscopy resulted in observed peaks at the 420 nm wavelength region of the spectrum. This result indicated and confirmed that 0.5 mM silver nitrate interacted with sesame oil cake extract to reduce Ag⁺ ions and achieve silver nanoparticles. The UV-visible spectrum was recorded at 48 h, 72 h, 96 h, 120 h, 144 h, and 168 h, and corresponds to 0.36, 0.39, 0.48, 0.52, 0.55, and 0.58 increasing absorbance values in the 420 nm region of the spectrum. This demonstrates increased interaction in the mixed solution, and increased nanoparticle synthesis over time. This also evidences the reaction between the 0.5 mM silver nitrate with sesame oil cake extract.

The resulting particle size was investigated at different time intervals for the changing composition of the reaction mixture. The reaction was carefully monitored every 24 hours for a solution of 0.5 mM AgNO₃. FIG. 2 shows a plot of the UV absorbance, at 420 nm wavelength, of 0.5 mM AgNO₃ interacted with sesame oil cake at time intervals from 48 h to 168 h. FIG. 2 demonstrates increased silver nanoparticles and increased absorbance over time. Specifically, FIG. 2 shows a linear increase in absorbance value at the specific spectrum region of 420 nm. These results further evidence increased interaction and synthesis of nanoparticles in the reaction mixture. FIG. 2 shows the results from time intervals ranging from 48 h to 168 h.

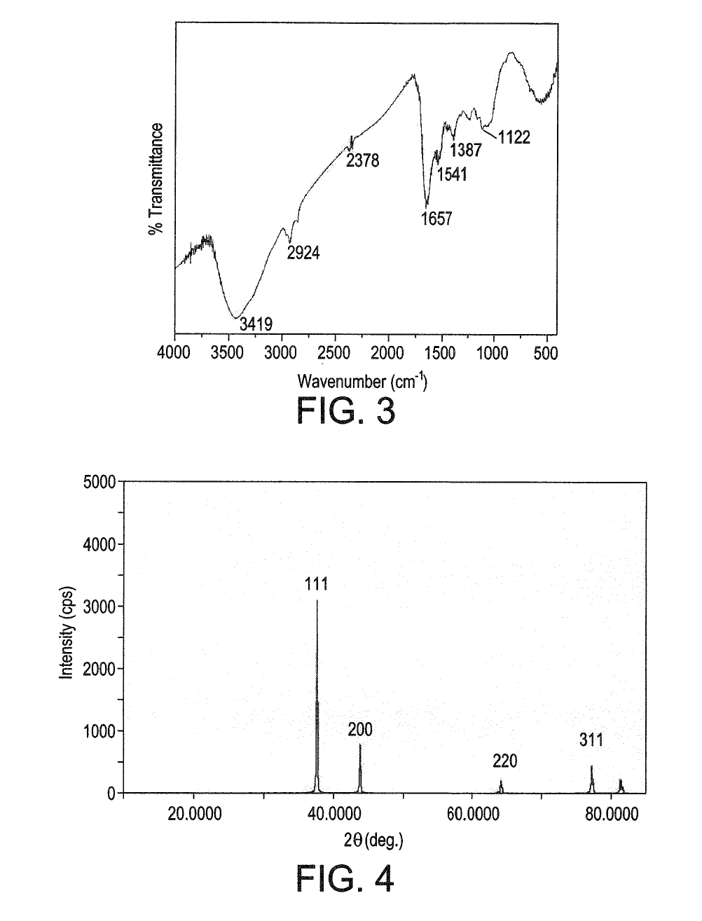

FIG. 3 shows the Fourier transform infrared spectroscopy (FTIR) spectrum obtained for the silver nanoparticles synthesized using sesame oil cake. The strong absorption peaks at 3419 cm⁻¹ result from stretching of the NH (amide) bond and the OH (alcohol) stretching. The absorption peak at 2924 cm⁻¹ is associated with the symmetrical stretching of a CH₂ group. The absorption peak at 2378 cm⁻¹ indicates the C═C═C stretching in the allene group. The peaks at 1657 and 1541 cm⁻¹ indicate the fingerprint region of C═O, C—O, and OH groups. The FTIR peak at 1387 cm⁻¹ identifies the amide I group, due to the carbonyl stretching vibration in amide linkages of the protein. The peak at 1122 cm⁻¹ is attributed to stretching of aliphatic amines. The FTIR spectrum is a useful tool for quantitative analysis of the reaction of specific silver nanoparticles. These results provide significant evidence of protein binding to the AgNPs, and also of the protein acting as reducing and stabilizing agent.

The X-Ray Diffraction Analysis (XRD) spectrum analysis of silver nanoparticles using sesame oil cake was performed on 0.5 mM silver nanoparticles to confirm the crystalline nature of the resulting structure. FIG. 4 shows the XRD spectrum of crystalline AgNPs synthesized using sesame oil cake. The XRD spectra demonstrated 20 values ranging from 20° to 80°. The XRD spectra confirms that the AgNPs exhibited at 20 values and reflection values of 38°, 47°, 65° and 80° corresponds to intensity (cps) of 111, 200, 220, and 311, respectively. The results confirm that the AgNPs produced from sesame oil cake have crystalline structure.

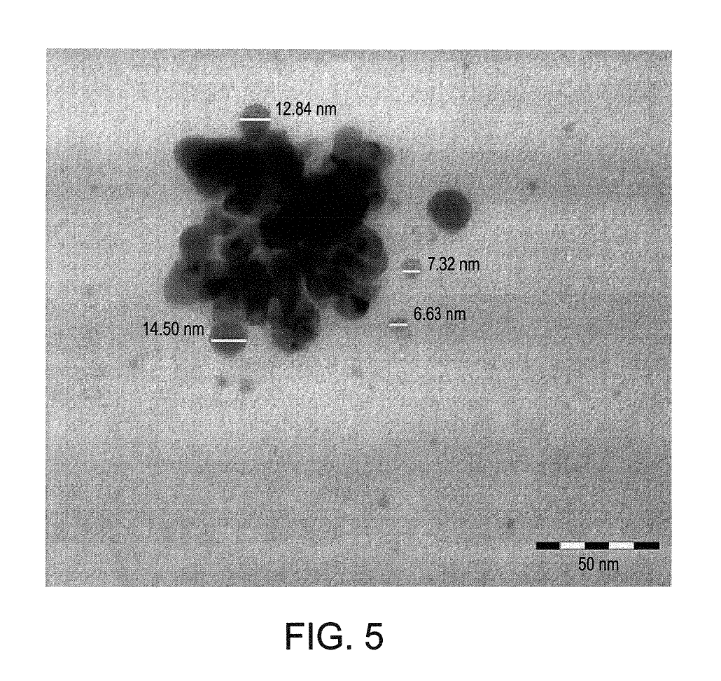

The synthesized AgNPs were dispersed in DH₂O for mixing under ultrasonic treatment. FIG. 5 shows a transmission electron microscopy (TEM) image of AgNPs synthesized using sesame oil cake. The TEM image shows the particle size ranges from 6.63 nm to 14.50 nm, with an average particle size of 10.32 nm. The TEM image shows that the AgNPs are mostly spherical in shape. This confirms that green or eco-friendly synthesis of small particle size AgNPs can be achieved by the present method. This small particle size is considered better for biological activity with regard to MCF-7 cancer cell lines. In fact, research has shown that smaller size nanoparticles achieve better physical and biological activity, e.g., better anticancer and antimicrobial properties.

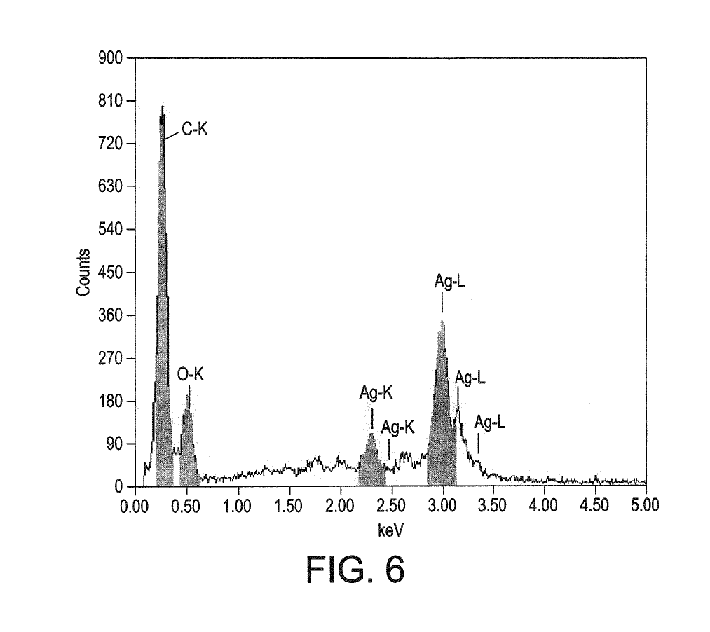

FIG. 6 shows Energy Dispersive X-ray diffraction (EDX) images of the synthesized AgNPs. The EDX analysis provides further confirmation that elemental silver is present in the synthesized nanoparticles. In the EDX images, all the peaks of silver are observed and indicated. In particular, the EDX images show peaks CK and K are from the grid and peaks of C, P and S correspond to the protein capping over the AgNPs. The intense signal of silver nanocrystals exhibited a peak at 3 keV. This is further evidence of successful synthesis of silver nanoparticles from sesame oil cake.

### Example 3

### Cytotoxicity Study

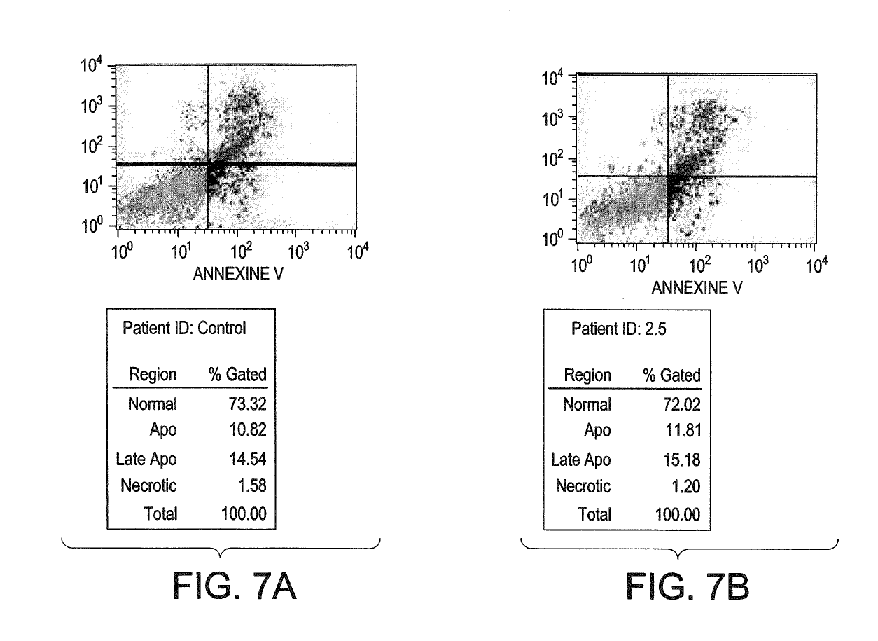

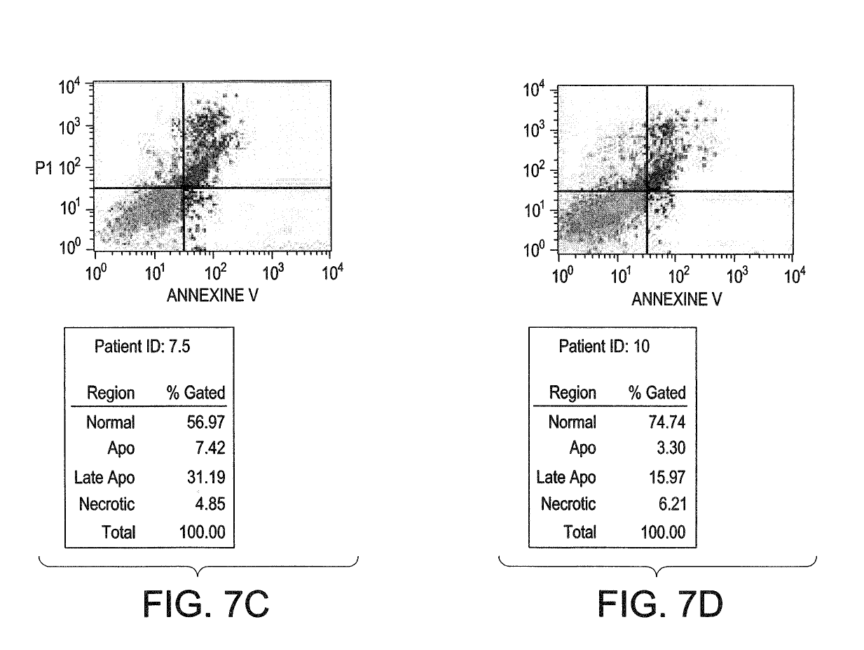

The cytotoxic effects of the synthesized AgNPs against human breast cancer cell line (MCF-7) were analyzed. FIGS. 7B-7D show the rate of apoptotic induction quantified by flow cytometry analysis. MCF-7 cells were plated in well plates for 24 h and then treated with AgNPs using sesame oil cake with concentrations of 2.5 μg/mL (FIG. 7B), 7.5 μg/mL (FIG. 7C) and 10 μg/mL (FIG. 7D) for an additional 24 h in a 5% CO₂ humidified atmosphere at 37° C. FIG. 7A shows data obtained for a control (MCF-7 cells without any treatment). At the end of the incubation period, the treated MCF-7 cells and controls were harvested and incubated with Annexin V and PI for 15 minutes before being analyzed on a flow cytometer with at 488 nm excitation and 515 nm for Annexin V detection and a filter with wavelengths above 600 nm for PI detection.

In the control, MCF-7 cells exhibited 14.54% late apoptosis and 10% early apoptosis. When the MCF-7 cells were treated with 2.5 μg/mL, 7.5 μg/mL, and 10 μg/mL of sesame oil cake derived AgNPs, the cells showed 15.18%, 31.19%, 15.97% late apoptosis, respectively, and 11.81%, 7.42%, 3.30% early apoptosis, respectively. These results show that the percentage of late apoptotic cells increased in all three concentrations. Among the three concentrations, MCF-7 cells treated with 7.5 μg/mL of sesame oil cake derived AgNPs were enhanced significantly, when compared to the other two concentrations and control groups. The number of early apoptotic cells decreased significantly in both 7.5 μg/mL and 10 μg/mL of sesame oil cake derived AgNPs compared to the control group. These results indicate that the sesame oil cake derived AgNPs enhanced cytotoxicity in MCF-7 cells. Therefore, the results show that using sesame oil cake derived AgNPs in higher concentrations regulates apoptotic induction.

Biological activities and other properties can be dependent on the size of the particles. The synthesized AgNPs have particle sizes in the range of 6.63 nm to 14.80 nm, and demonstrated better results in human breast carcinoma cancer cell line (MCF-7).

It is to be understood that the present subject matter is not limited to the embodiments described above, but encompasses any and all embodiments within the scope of the following claims.

1. A method of producing silver nanoparticles using Sesame indicum oil til cake after the Sesame indicum oil til cake has been washed to remove additives used in the Sesame indicum oil extraction process, comprising:
providing a room-temperature dried Sesame indicum oil til cake, the Sesame indicum oil til cake being from the Makkah region of Saudi Arabia;
grinding the sesame oil til cake to a fine powder;
soaking the sesame oil til cake powder in distilled water for 24 hours;
filtering the sesame oil til cake powder to produce sesame oil cake extract;
adding distilled water to silver nitrate to obtain a silver nitrate solution;
adding the silver nitrate solution to the sesame oil cake extract to provide a mixed solution; and
maintaining the mixed solution for a period of time sufficient for formation of the silver nanoparticles, wherein the silver nanoparticles have a crystalline structure, are spherical in shape, and have an average particle size of 10.32 nm.

2-5. (canceled)

6. The method of claim 1, wherein the silver nanoparticles form after the solution is maintained for a period of at least about 48 hours.

7. The method of claim 6, wherein the silver nanoparticles form after the mixed solution is maintained at room temperature for a period of at least about 96 hours.

8. The method of claim 7, wherein the silver nanoparticles form after the mixed solution is maintained at room temperature for a period of at least about 144 hours.

9. The method of claim 8, wherein the silver nanoparticles from after the mixed solution is maintained at room temperature for a period of at least about 168 hours.

10. The silver nanoparticles produced by the method of claim 1.

11-13. (canceled)

14. A method of treating cancer in a patient using an effective amount of the silver nanoparticles of claim 11.

15. A method of treating cancer in a patient using an effective amount of the silver nanoparticles of claim 12.

16. The method of claim 15 wherein the cancer is breast cancer.

17-18. (canceled)
